(19) United States
(12) Patent Application Publication
(10) Pub. No.: US 2003/0162996 A1
(43) Pub. Date: Aug. 28, 2003

(54) Method for isolating polymethyl alkanoic acids and disposal of the resulting wastes

(76) Inventors: Hagen Weigl, Ladenburg (DE); Klaus Ebel, Lampertheim (DE); Gerhard Schulz, Ludwigshafen (DE)

Correspondence Address: KEIL & WEINKAUF, 1350 CONNECTICUT AVENUE, N.W., WASHINGTON, DC, 20036, US

(21) Appl. No.: 10/296,254

(22) PCT Filed: May 21, 2001
(86) PCT No.: PCT/EP01/05816

(30) Foreign Application Priority Data
May 26, 2000 (DE) 10026134.5

(51) Int. Cl. C07C 51/235; C07C 51/16

(52) U.S. Cl. 562/531

(57) ABSTRACT

A process for isolating polymethylolalkanoic acids or monomethylolalkanoic acids of the formula (I)

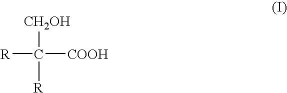

where R are identical or different and are each a substituted or unsubstituted hydrocarbon radical or a methylol group, which have been prepared from the corresponding polymethylolalkanals or monomethylolalkanals of the formula (II)

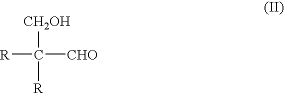

where R are as defined above, by oxidation using an aqueous solution of hydrogen peroxide comprises

a) carrying out a crystallization followed by a liquid/solid separation (B) to give the acid as first crystallized material (2a) plus mother liquor (3),

b) producing further crystallized material (2b) in the mother liquor (3) and separating the resulting mother liquor (3) from the further crystallized material (2b) in a further solid/liquid separation (C), and

c) disposing of the resulting mother liquor (3) which has been depleted in the acid.

The present invention relates to a process for isolating polymethylolalkanoic acids or monomethylolalkanoic acids and preferably dimethylolalkanoic acids.

The preparation of polymethylolalkanoic acids or dimethylolalkanoic acids by oxidation of the corresponding aldehydes to the carboxylic acid is generally known. In these known processes, water serves as solvent. As a result, considerable amounts of mother liquor and wastewater and also washing water are obtained, presenting disposal problems. Disposal can be carried out either by passing all the waste streams into a water treatment, complicated separations by means of ion exchangers to reduce the concentration of the desired end product or of by-products such as formic acid or by targeted work-up. U.S. Pat. No. 4,676,912 of International Minerals & Chemical Corporation describes a work-up as microemulsion specifically for dimethylolpropionic acid which has been prepared as described in U.S. Pat. No. 3,312,736.

A disadvantage of all known processes is that the disposal of the mother liquor from the crystallization and the washing water via a water treatment plant represents a high degree of environmental pollution, particularly because of the high residual solubility of, for example, dimethylolpropionic acid relative to the acid recovered by crystallization. Although it has been proposed in the prior art that the proportion of desired acid isolated be increased by separations over ion exchangers, this has the disadvantage that the wastewater streams obtained remain equally large and regeneration streams are obtained in addition. The work-up as microemulsion, as proposed in U.S. Pat. No. 4,676,912, is also very complicated and incurs high costs.

It is an object of the present invention to provide a process for isolating polymethylolalkanoic acids or monomethylolalkanoic acids and particularly dimethylolalkanoic acids, in which the solvent used has, after isolation of the acid, only a minimal content of this acid and/or by-products and at the same time enables optimal disposal of any by-products or residues and also makes possible an increase in the yield of the desired acid.

We have found that this object is achieved by a process for isolating polymethylolalkanoic acids or monomethylolalkanoic acids of the formula (I)

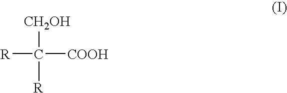

where R are identical or different and are each a substituted or unsubstituted hydrocarbon radical or a methylol group, which have been prepared from the corresponding polymethylolalkanals or monomethylolalkanals of the formula (II)

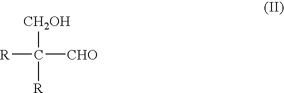

where R are as defined above, by oxidation using an aqueous solution of hydrogen peroxide, which comprises

a) carrying out a crystallization followed by a liquid/solid separation to give the acid as first crystallized material plus mother liquor,

b) producing further crystallized material in the mother liquor and separating the resulting mother liquor from the further crystallized material in a further solid/liquid separation, and

c) disposing of the resulting mother liquor which has been depleted in the acid.

The reaction mixture in the preparation of polymethylolalkanoic acids or monomethylolalkanoic acids, in particular dimethylolalkanoic acids, consists essentially of an aqueous solution of the corresponding alkanals and other organic compounds as secondary components and an aqueous solution of hydrogen peroxide which serves as oxidant. The crystallization of the desired polymethylolalkanoic acid, also referred to as end product for the purposes of the present invention, is followed by a solid/liquid separation which gives a crystalline end product and a filtrate which is also referred to as mother liquor for the purposes of the present invention.

In an embodiment of the process of the present invention, the mother liquor obtained after step a) is allowed to crystallize by cooling in a step a1) to form a further crystallized material and further mother liquor.

In a further process step a2), solvent or a solvent mixture is firstly at least partly removed by vaporization from the mother liquor obtained in step a) of claim 1 or a1) of claim 2.

In this way, a higher proportion of desired acid is obtained and, secondly, the major part of the solvent can subsequently be discharged as an only slightly contaminated distillate which can thus be passed to a water treatment plant. This distillate has a TOC content of usually <25%, preferably <15%, particularly preferably <10%. The residue which remains has a high calorific value and is therefore suitable for residue incineration. This residue has a water content of usually <70%, preferably <60%, particularly preferably <50%.

If solvent, e.g. water, is removed from the reaction mixture by vaporization, this can be carried out either at atmospheric pressure, at superatmospheric pressure or under reduced pressure. Preference is given to atmospheric pressure or reduced pressure. Setting of a defined temperature between the solidification point and the boiling point of the reaction mixture, preferably in the range from 0° C. to 55° C., leads to the major part of the desired polymethylolalkanoic acid crystallizing out. This is, however, also possible without prior distillation.

The first crystallized material can then be washed with a suitable solvent or solvent mixture.

Each further crystallized material obtained can be dissolved in a solvent or solvent mixture and passed to step a) of claim 1. Furthermore, the washing liquid obtained from washing of the first crystallized material can be allowed to crystallize and be subjected to a solid/liquid separation, producing further crystallized material. The resulting mother liquor is disposed of. The solvent or solvent mixture for dissolving the further crystallized material can also be the washing liquid, i.e. it can already contain polymethylolalkanoic acid.

The washing liquid formed in the washing of the first crystallized material can also, in a further embodiment of the process of the present invention, be passed to step a) of claim 1 or be combined with the mother liquor obtained from step a).

In a further embodiment b1), the further crystallized material formed after step b) of claim 1 can be passed to the solid/liquid separation of step a) of claim 1.

The modes of operation described, including their alternative embodiments, have the advantage that any solids treatment in which no end product from the first crystallization participates is dispensed with and no product of inferior quality is obtained.

The procedures described can be repeated until no more end product, i.e. first crystallized material, is obtained. The filtrate obtained to this point in time is then greatly depleted in solvent, preferably water, and can be passed directly to disposal, e.g. incineration. Advantageously, the liquid stream is only a fraction of the original waste stream and, in addition, any incineration process provided for disposal is very environmentally friendly and also inexpensive. The remainder of the liquid stream, which may still contain washing water used, is obtained as distillate during the course of the process. This represents only slightly contaminated wastewater having a significantly reduced TOC content and can be treated appropriately in a water treatment plant without problems and without ecological disadvantages.

The process of the present invention, preferably supplemented by removal of solvent by distillation either during the oxidation reaction or immediately afterward, therefore makes possible a higher yield of first crystallized material and thus of end product and at the same time allows easier disposal of the remaining organic by-products by means of incineration and of only slightly contaminated solvent via a water treatment plant.

Preference is given to using water as solvent and the polymethylolalkanoic acid which is preferably isolated by means of the process is dimethylolpropionic acid.

The invention is illustrated below by means of illustrative embodiments and the accompanying drawing.

In the drawing:

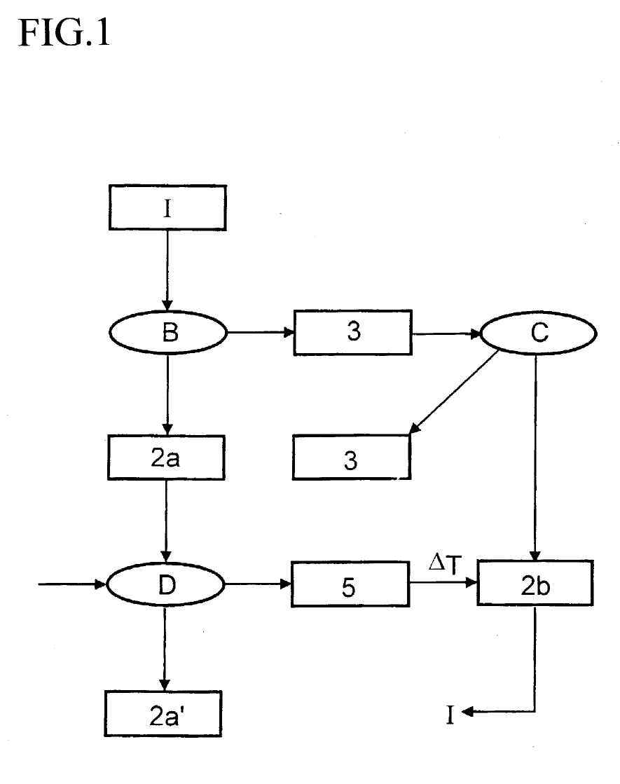

FIG. 1: shows a flow diagram of a first illustrative embodiment of the process of the present invention,

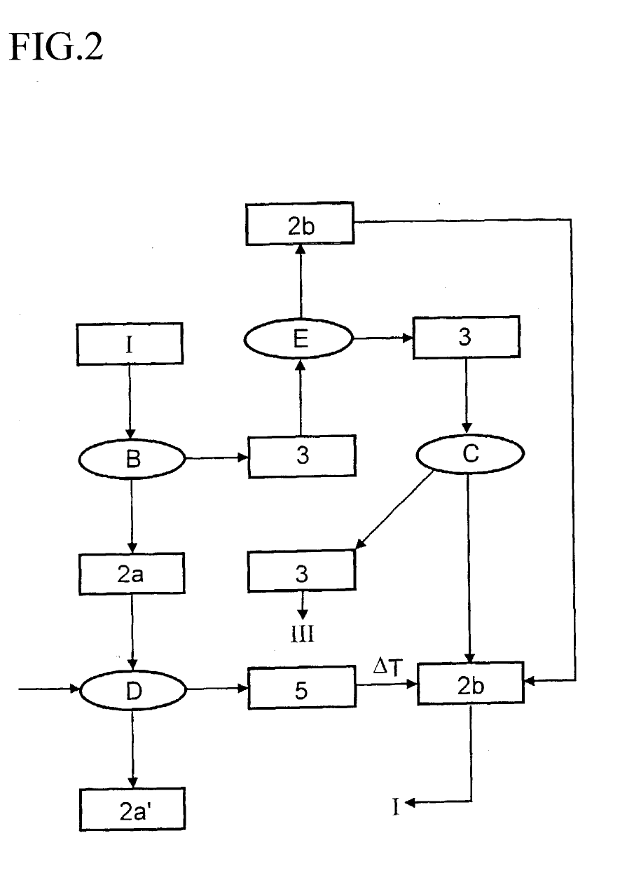

FIG. 2: shows a flow diagram of a second illustrative embodiment of the process of the present invention and

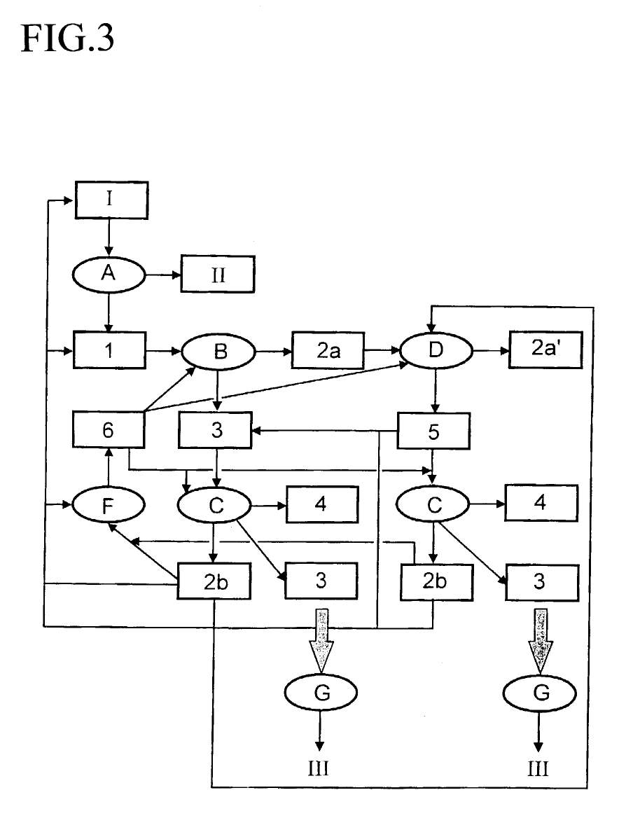

FIG. 3: shows a flow diagram of a third illustrative embodiment of the process of the present invention.

FIG. 1 shows the flow diagram of a first illustrative embodiment in which the reaction product comprising a polymethylolalkanoic or monomethylolalkanoic acid prepared by oxidation using an aqueous solution of hydrogen peroxide is denoted by I. This reaction product (I) is fed to a crystallization followed by a solid/liquid separation (B), which results in, firstly, the desired polymethylolalkanoic or monomethylolalkanoic acid end product as first crystallized material (2 a) and, secondly, mother liquor (3). Renewed concentration by evaporation (C) again gives crystallized material, now denoted as second or further crystallized material (2b), and mother liquor (3). The desired pure end product, which here is only the crystallized material denoted by (2a), is purified by washing (D), with the washing water (5) obtained serving, with or without input of heat, to dissolve the second crystallized material (2b) also obtained. This solution is fed back into the reaction product (I) to increase the proportion of desired pure end product (2a). The remaining mother liquor (3) is passed to incineration (III). If desired, the mother liquor (3) can be further concentrated prior to incineration (III), and the distillate formed, for example, in the concentration step is only very slightly contaminated and can be passed to a water treatment plant. This alternative is not shown in more detail in FIG. 1.

FIG. 2 shows a modification of the above-described mode of operation of the first illustrative embodiment; only the additional process steps carried out according to this variant will be described below. As a deviation from the first illustrative embodiment, the mother liquor (3) obtained after solid/liquid separation (B) of the reaction product (I) is cooled in an intermediate step (E), as a result of which further crystallized material (2b) crystallizes out and further mother liquor (3) is also obtained. This intermediate step achieves further depletion of the mother liquor (3) by recovery of additional crystallized material (2b). The mother liquor (3) obtained after this intermediate step (E) is then again depleted by evaporation (C) to give further crystallized material (2b) and can then be passed to incineration (3), if desired supplemented by concentration by evaporation and passing the resulting distillate to a water treatment plant, as previously indicated for the first illustrative embodiment but not shown either there or here in the figure. Both further crystallized materials (2b) can then be combined and dissolved by means of the washing water (5) from the first crystallized material from step (D), with or without input of heat, and fed back to the reaction product (I).

FIG. 3 shows a flow diagram which depicts a further embodiment of the process of the present invention. Here, (I) denotes the reaction product obtained in the reaction of dimethylolalkanal or another polymethylolalkanal or monomethylolalkanal with an aqueous solution of hydrogen peroxide. This reaction product (I) is firstly concentrated by evaporation (A), e.g. by means of a falling film evaporator at atmospheric pressure or under reduced pressure. If desired, this step can be carried out during the oxidation procedure itself, at atmospheric pressure or under reduced pressure. Water (II) is subsequently separated off and the resulting reaction mixture (1) which has been depleted in water is passed to a solid/liquid separation (B) which gives, firstly, the desired end product (2a), e.g. dimethylolpropionic acid, and, secondly, mother liquor (3) or filtrate from the solid/liquid separation (B). By means of renewed concentration by evaporation and solid/liquid separation (C), solvent (4), which can be water or any other suitable solvent or a solvent mixture, is again separated off and end product (2b) and mother liquor (3) are again obtained. In a continuous process as shown in the flow diagram, the further crystallized material (2b) is either recycled to the reaction product (I) or the reaction mixture (1) resulting after the evaporation (A) or is dissolved in a suitable solvent or solvent mixture (F) and used as washing liquor (6) in the solid/liquid separation (B), (C) or (C'). This leaves the mother liquor (3) which, as a result of the indicated process procedure, represents a residue having a high calorific value and can be passed to incineration (III). However, it can be concentrated beforehand in a process step (G). All the distillation streams mentioned above or below which are produced by concentration or evaporation are unproblematical and can thus be passed to a water treatment plant.

The end product (2a) resulting from the solid/liquid separation (B) is washed with a suitable solvent, preferably water, in the region denoted by (D) in the flow diagram and gives the desired purified end product (2a), with the washing liquid, preferably the washing water (5), also being obtained. This liquid is again concentrated and passed to a solid/liquid separation (C), giving the solvent, e.g. water (4), and further crystallized material (2b) which is either recycled to the reaction mixture (1) or the reaction product (I) or is dissolved in a suitable solvent or solvent mixture (F) and used as washing liquor (6) in a solid/liquid separation (B) or (C). This leaves mother liquor (3) which can be passed to incineration (III), if desired once more after prior evaporation in step (G).

As an alternative, the washing water (5) which has been obtained after the washing step (D) can be combined with the mother liquor (3) obtained from the solid/liquid separation (B) in order to be evaporated together with the latter and passed to a solid/liquid separation (C) which in turn gives a solvent or water (4), the mother liquor (3) to be passed to incineration (III) and end product (2b).

Alternatively, the washing water (5) which has been obtained after the washing step (D) can be used for dissolving the end product (2b) which has been obtained by evaporation and solid/liquid separation (C) of the mother liquor, possibly at elevated temperature, in order to be then recycled as solution of (2b) in (5) to step (I) or (1) of the work-up process.

The solvent or water (4) is, as analyses show, only very slightly contaminated and can readily be passed to a water treatment plant. This is no longer shown in the flow diagram. The mother liquor (3) represents a residue which can readily be incinerated and the end product (2a) can be obtained in high purity and in a yield greater than that in conventional processes.

In the following examples, the letters and numbers in brackets correspond to those in the flow diagrams.

### EXAMPLE 1

614.4 g of an aqueous reaction solution (I) containing 1.45 mol of dimethylolpropionic acid is evaporated (A) to 382.2 g and gives a reaction mixture (1). After the solid/liquid separation (B) at 4° C., 199.3 g of mother liquor (3) and 153.4 g of dimethylolpropionic acid (dry, 84.7% pure) (2a) are obtained. 59.1 g of distillate (4) are again taken off from the mother liquor (3), and dimethylolpropionic acid is then separated off as solid (2b) at 4° C. The yield of dimethylolpropionic acid (2b) (60.6% pure) is 14.3 g. The reaction is carried out batchwise, so that the end product (2b) as indicated in the flow diagram corresponds to the end product (2a) obtained.

### EXAMPLE 2

656 g of a reaction mixture (I) from the reaction of dimethylolpropionaldehyde (aqueous) with hydrogen peroxide are concentrated (A) to 481 g by distillation and give reaction mixture (1) (35% strength by weight dimethylolpropionic acid). A solid/liquid separation of the suspension (1) obtained gives 140 g of dimethylolpropionic acid (dry, 79.9% pure) (2a) and 199.5 g of mother liquor (3). Of this, 185 g are evaporated to 142 g and, after solid/liquid separation (C) at 4° C., give 19.7 g of dimethylolpropionic acid (2b=2a) and 112 g of filtrate (3). If 105 g of this are evaporated to 56 g, a residue having a TOC content of 44.6% is obtained.

### EXAMPLE 3

45 kg of distillate (II) are taken off from 150 kg of a reaction product (I) from the reaction of dimethylolaldehyde and hydrogen peroxide. Filtration of the resulting suspension (1) (31% strength by weight dimethylolpropionic acid) gives 60.1 kg of mother liquor (3) and, after washing with water (D), 32.7 kg of dimethylolpropionic acid (moist, 69.6% pure) (2a). The washing water (31.1 kg) (5) and the mother liquor (3) are combined and 54 kg of distillate (4) are taken off. Solid/liquid separation (C) gives 27.2 kg of filtrate or mother liquor (3) and 10.1 kg of dimethylolpropionic acid (moist, 67.6% pure) (2b=2a).

### EXAMPLE 4

36.8 kg of distillate (II) are taken off from 142.6 kg of a completely reacted reaction product (I) from the reaction of dimethylolaldehyde and hydrogen peroxide. The bottoms (1) (32% strength by weight dimethylolpropionic acid) which remain are cooled and subjected to solid/liquid separation (B). The solid (2a) obtained is washed with water (D). This gives 34.5 kg of dimethylolpropionic acid (moist, 55.3% pure) (2a). The filtrate (3) has a water content of 69% (C: 15%, O: 73%, H: 10%, N: 1%). Another 30.4 kg of distillate (4) are taken off from this by evaporation (C) and 3.1 kg of solid (2b) and 15.8 kg of filtrate (3) are obtained (C: 33%, O: 55%, H: 10%, N: 2%). The 2nd crystallized material (2b) is, together with the washing water (5) from the first crystallized material (2a), recycled to the next batch (I). From this, dimethylolpropionic acid (2a) is isolated in a total yield of 63%. Analogous batches without recirculation give, on average, isolated total yields of 50%.

We claim:

1. A process for isolating polymethylolalkanoic acids or monomethylolalkanoic acids of the formula (I) 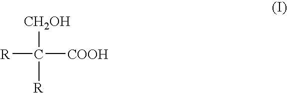
where R are identical or different and are each a substituted or unsubstituted hydrocarbon radical or a methylol group, which have been prepared from the corresponding polymethylolalkanals or mono-methylolalkanals of the formula (II) 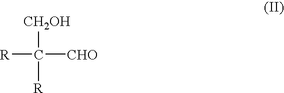
where R are as defined above, by oxidation using an aqueous solution of hydrogen peroxide, which comprises
a) carrying out a crystallization followed by a liquid/solid separation (B) to give the acid as first crystallized material (2a) plus mother liquor (3),
b) producing further crystallized material (2b) in the mother liquor (3) and separating the resulting mother liquor (3) from the further crystallized material (2b) in a further solid/liquid separation (C), and
c) disposing of the resulting mother liquor (3) which has been depleted in the acid.

2. A process as claimed in claim 1, wherein
a1) the mother liquor (3) obtained after step a) is allowed to crystallize by cooling to form a further crystallized material (2b) and further mother liquor (3).

3. A process as claimed in claim 1 or 2, wherein
a2) solvent or a solvent mixture is firstly at least partly removed by vaporization from the mother liquor (3) obtained in step a) of claim 1 or a1) of claim 2.

4. A process as claimed in any of claims 1 to 3, wherein
d) the first crystallized material (2a) is purified by washing.

5. A process as claimed in claim 4, wherein
e) each further crystallized material (2b) is dissolved in a solvent or solvent mixture and passed to step a) of claim 1.

6. A process as claimed in claim 4 or 5, wherein f1)the washing liquid (5) obtained from washing of the first crystallized material (2a) can be allowed to crystallize and be subjected to a solid/liquid separation (C), producing further crystallized material (2b), and the resulting mother liquor (3) is disposed of.

7. A process as claimed in claim 4, wherein f2)the washing liquid (5) formed in the washing of the first crystallized material (2a) is passed to step a) of claim 1 or is combined with the mother liquor (3) obtained from step a).

8. A process as claimed in claim 1, wherein b1)the further crystallized material (2b) formed after step b) of claim 1 is passed to the solid/liquid separation (B) of step a) of claim 1.

9. A process as claimed in any of claims 1 to 8, wherein solvent or a solvent mixture is at least partly removed from the reaction mixture by vaporization during the oxidation reaction or immediately afterward.

10. A process as claimed in any of claims 1 to 9, wherein water is used as solvent.

11. A process as claimed in any of claims 1 to 10 for isolating dimethylolalkanoic acid, preferable dimethylolpropionic acid.
